(19) United States
(12) Patent Application Publication
(10) Pub. No.: US 2024/0189214 A1
(43) Pub. Date: Jun. 13, 2024

(54) Silicone Mixture for Hair Conditioning

(71) Applicant: The Procter & Gamble Company, Cincinnati, OH (US)

(72) Inventors: Chunyan Zhang, Singapore (SG); Robert Wayne Glenn, JR., Singapore (SG)

(21) Appl. No.: 18/522,301

(22) Filed: Nov. 29, 2023

Related U.S. Application Data
(60) Provisional application No. 63/428,427, filed on Nov. 29, 2022.

(51) Int. Cl. A61K 8/898; A61Q 5/12

(52) U.S. Cl. CPC A61K 8/898; A61Q 5/12; A61K 2800/413; A61K 2800/5426

(57) ABSTRACT

A hair conditioner composition the exploits a blend of a polyquaternium silicone nanoemulsion and a silicone oil to provide improved conditioning benefits and composition stability. The composition may also include a cationic surfactant, a fatty alcohol, and an aqueous carrier. When the polyquaternium silicone nanoemulsion and silicone oil are blended at the right ratio, the composition provides improved hair free flow and exhibits good stability.

### FIELD

The present disclosure relates generally to a hair conditioning composition that includes a blend of a silicone nanoemulsion and a silicone oil. More specifically, the present disclosure is directed to a hair conditioning composition that contains a blend of a polyquaternium silicone nanoemulsion and a silicone oil and exhibits improved conditioning and product stability.

### BACKGROUND

Conventional hair conditioners commonly contain silicone polymers to provide a dry conditioning benefit, as well as high levels of high melting point fatty compounds (e.g., C16 to C18 fatty alcohols), which act as structuring agents wherein combined with a suitable surfactant and an aqueous carrier to form a gel network. A gel network provides a viscous and high yield point rheology which facilitates the dispensing of the conditioner from a bottle or tube and the subsequent distribution and spreading of the product through the hair by a consumer. Gel network structuring also enables incorporation of other common ingredients such as silicones, perfumes, and oils in the form of an oil-in-water emulsion that is shelf stable. These silicones and oils are intended to be deposited on the hair to provide the primary hair conditioning benefits, including wet and dry combing friction reduction and hair manageability.

However, silicone polymers can build up on hair over multiple uses, and the high melting point fatty compounds may co-deposit with silicone, leading to an undesirable waxy buildup on hair. Indeed, a major consumer complaint with hair conditioners is waxy residue that makes hair look greasy or feel heavy.

Nanoemulsion silicones can be used to provide a cleaner hair feel with less buildup but may exhibit reduced conditioning efficacy and less conditioner stability (e.g., visible phase separation). Accordingly, it would be desirable to provide a hair conditioner with a nanoemulsion silicone that provides good conditioning and has good stability.

### SUMMARY

Disclosed herein is a hair conditioner composition comprising a blend of a polyquaternium silicone nanoemulsion and a silicone oil. The polyquaternium silicone contained in the nanoemulsion has a particle size of 1 nm to 100 nm and includes silicone blocks of 80 to 250 siloxane units. The silicone oil has a particle size of 1 micron to 100 microns. The ratio of the polyquaternium silicone to silicone oil in the blend ranges from 1:2 to 3:1. The hair conditioner composition may optionally include a cationic surfactant, a fatty alcohol and an aqueous carrier that form a gel network.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 shows the microscopy of silicone distribution of inventive and comparative example conditioners; and

FIG. 2 shows SEM images of hair treated with inventive and comparative example formulas.

### DETAILED DESCRIPTION

The present inventors have discovered that use of a silicone nanoemulsion blended with a silicone oil provides improved hair free flow while still preserving high conditioning and being a stable product. Surprisingly, it has also been discovered that blending a polyquaternium silicone nanoemulsion and a silicone oil at a particular ratio and incorporating the blend into a hair conditioner composition yields improved dry conditioning without sacrificing product stability.

Reference within the specification to “embodiment(s)” or the like means that a particular material, feature, structure and/or characteristic described in connection with the embodiment is included in at least one embodiment, optionally, a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the composition, unless specifically stated otherwise, and may be designated as “wt %.” All ratios are weight ratios, unless specifically stated otherwise. All ranges are inclusive and combinable. The number of significant digits convey neither a limitation on the indicated amounts nor on the accuracy of the measurements. Unless otherwise indicated, all measurements are understood to be made at 25° C. and at ambient conditions, where “ambient conditions” means conditions under 1 atmosphere of pressure and at 50% relative humidity. All numeric ranges are inclusive of narrower ranges that fall within such broader numerical range, as if such narrower numerical ranges were all expressly written herein. Delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well (e.g., “one or more”), unless the context clearly indicates otherwise.

“Molecular weight” or “MW” or “M.Wt.” refers to weight average molecular weight, unless otherwise stated.

“Includes” and variations thereof are meant to be non-limiting and are understood to mean “comprises.”

“Nanoemulsion” means an oil-in-water (o/w) emulsion with an average particle size of 1 nm to 100 nm. The particle size referred to herein is z-average measured by dynamic light scattering. The nanoemulsions described herein may be prepared by: (1) mechanically breaking down the emulsion droplet size; (2) spontaneously forming an emulsion, which may be referred to as a microemulsion in the literature; and (3) using emulsion polymerization to achieve average particle size in the target range described herein.

Unless otherwise noted, all component or composition levels are in reference to the active portion of that component or composition, and are exclusive of impurities, for example, residual solvents or by-products, which may be present in commercially available sources of such components or compositions.

### Silicones

The silicones in this invention comprise two types; one type is the nano-sized silicone droplets contained inside the silicone nanoemulsion, and another type is the neat silicone oil. The percentage disclosed below is the total silicone composition adding both types together.

The hair care composition may include 0.5% to 18% (e.g., 3% to 18%, 4% to 16%, 5% to 14%, 6% to 12%, 6% to 10%, 3% to 8%, or even 2% to 6%) of one or more silicones by weight of the hair care composition.

### Silicone Oil

The one or more silicones may include silicone oils that have in their molecular structure polar functional groups such as Si—OH (present in dimethiconols), primary amines, secondary amines, tertiary amines, and quaternary ammonium salts. The one or more silicones may be selected from the group consisting of aminosilicones, pendant quaternary ammonium silicones, terminal quaternary ammonium silicones, amino polyalkylene oxide silicones, quaternary ammonium polyalkylene oxide silicones, and amino morpholino silicones.

The one or more silicones can include one or more aminosilicones corresponding to formula (I):

RₐG₃₋ₐ—Si(OSiG₂)ₙ—(OSiG_{b}R_{2-b})ₘ—O—SiG₃₋ₐ—Ra (I)

in which:
G is chosen from a hydrogen atom, a phenyl group, OH group, and C₁-C₈ alkyl groups, for example methyl,
a is an integer ranging from 0 to 3, and alternatively a is 0,
b is chosen from 0 and 1, and alternatively b is 1,
m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10;
R′ is a monovalent group of formula —C_{q}H_{2q}L in which q is a number from 2 to 8 and L is an optionally quaternized amine group chosen from the groups:

—NR′—CH₂—CH₂—N′(R¹)₂,

—N(R″)₂,

—N⁺(R″)₃A⁻,

—N⁺H(R″)₂A⁻,

−N⁺H₂(R″)A⁻, and

—N(R″)—CH₂—CH₂—N⁺R″H₂A⁻,

in which R″ can be chosen from a hydrogen atom, phenyl groups, benzyl groups, and saturated monovalent hydrocarbon-based groups, such as for example an alkyl group comprising from 1 to 20 carbon atoms, and A⁻ is chosen from halide ions such as, for example, fluoride, chloride, bromide and iodide.

The one or more silicones may include those corresponding to formula (1) wherein a=0, G=methyl, m and n are numbers such that the sum (n+m) can range for example from 1 to 2 000, such as for example from 50 to 150, wherein n can be for example chosen from numbers ranging from 0 to 1 999, such as for example from 49 to 149, and wherein m can be chosen from numbers ranging for example from 1 to 2 000, such as for example from 1 to 10; and L is —N(CH₃)₂ or —NH₂, alternatively —NH₂.

The one or more silicones can include pendant quaternary ammonium silicones of formula (II):

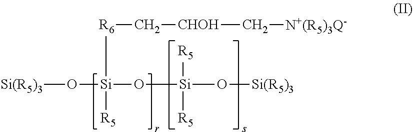

in which:
R₅ is chosen from monovalent hydrocarbon-based groups comprising from 1 to 18 carbon atoms, such as C₁-C₁₈ alkyl groups and C₂-C₁₈ alkenyl groups, for example methyl;
R₆ is chosen from divalent hydrocarbon-based groups, such as divalent C₁-C₁₈ alkylene groups and divalent C₁-C₁₈ alkylenoxy groups, for example C₁-C₈ alkylenoxy groups, wherein said R₆ is bonded to the Si by way of a Si—C bond;
Q⁻ is an anion that can be for example chosen from halide ions, such as chloride, and organic acid salts (such as acetate);
r is an average statistical value ranging from 2 to 20, such as from 2 to 8;
s is an average statistical value ranging from 20 to 200, such as from 20 to 50.

Such aminosilicones are described more particularly in U.S. Pat. No. 4,185,087, the disclosure of which is incorporated by reference herein.

A silicone which falls within this class is the silicone sold by the company Union Carbide under the name “Ucar Silicone ALE 56”.

Further examples of the one or more silicones include quaternary ammonium silicones of formula (III):

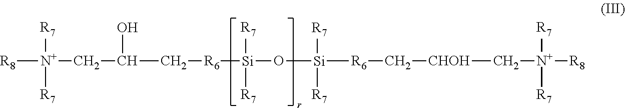

in which:
groups R₇, which may be identical or different, are each chosen from monovalent hydrocarbon-based groups comprising from 1 to 18 carbon atoms, such as C₁-C₁₈ alkyl groups, for example methyl, C₂-C₁₈ alkenyl groups, and rings comprising 5 or 6 carbon atoms;
R₆ is chosen from divalent hydrocarbon-based groups, such as divalent C₁-C₁₈ alkylene groups and divalent C₁-C₁₈alkylenoxy, for example C₁-C₈, group connected to the Si by a Si—C bond;
R₈, which may be identical or different, represent a hydrogen atom, a monovalent hydrocarbon-based group comprising from 1 to 18 carbon atoms, and in particular a C₁-C₁₈ alkyl group, a C₂-C₁₈ alkenyl group or a group —R₆—NHCOR₇;
X⁻ is an anion such as a halide ion, in particular chloride, or an organic acid salt (acetate, etc.); r represents an average statistical value from 2 to 200 and in particular from 5 to 100.

Such silicones are described, for example, in application EP-A-0 530 974. Silicones falling within this class can be the silicones sold by the company Goldschmidt under the names Abil Quat 3270, Abil Quat 3272 and Abil Quat 3474. Further examples include quaternary ammonium and polyalkylene oxide silicones, wherein the quaternary nitrogen groups are located in the polysiloxane backbone, at the termini, or both. Such silicones are described in PCT Publication No. WO 2002/010257. Silicones falling within this class include silicones sold by the company Momentive under the name Silsoft Q™.

The one or more silicones can include aminofunctional silicones having morpholino groups of formula (IV):

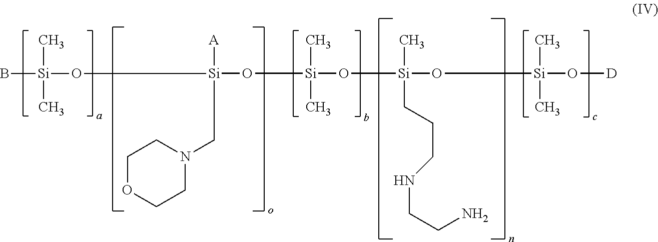

in which
A denotes a structural unit (I), (II), or (III) bound via —O—

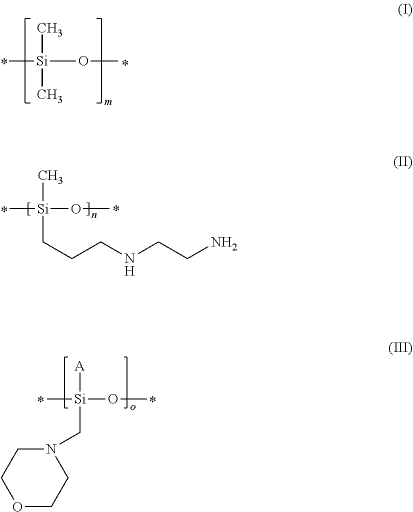

or an oligomeric or polymeric residue, bound via —O—, containing structural units of formulas (I), (II), or (III), or half of a connecting oxygen atom to a structural unit (III), or denotes —OH,
* denotes a bond to one of the structural units (I), (II), or (III), or denotes a terminal group B (Si-bound) or D (O-bound),
B denotes an —OH, —O—Si(CH₃)₃, —O—Si(CH₃)₂OH, or —O—Si(CH₃)₂OCH₃ group,
D denotes an —H, —Si(CH₃)₃, —Si(CH₃)₂OH, or —Si(CH₃)₂OCH₃ group,
a, b, and c denote integers between 0 and 1000, provided that a+b+c>0,
m, n, and o denote integers between 1 and 1000.

Aminofunctional silicones of this kind can bear the INCI name: Amodimethicone/Morpholinomethyl Silsesquioxane Copolymer. Some commercially available examples of silicones that may be suitable for use herein include 2-8566, AP 6087, AP 6088, DC 8040, 8822A DC, DC 8803 & 8813 polymer, 7-6030, AP-8104, AP 8201, CE-8170 AF, 2-8177, 2-8194, 9224, 939, 949, 959, DC 5-7113, DC 5-7070, DC CE-8810, CE 8401, CE 1619, SS-3551 and SS-3552 from Dow Corning; ADM 652, ADM 656, WR 1100, 1300, 1650 (fluids), ADM 6060 linear amodimethicone emulsion, ADM 6057 E branched amodimethicone emulsion, ADM 8020 VP micro emulsion and SLM 28040 micro emulsion from Wacker; Silsoft® 331, SF1708, SME 253 & 254 emulsion, SM2125 emulsion, SM 2658 emulsion, and Silsoft® Q emulsion from Momentive; KF-889, KF-867S, KF-8004, and X-52-2265 emulsion from Shin Etsu; Siltech® E-2145 and E-Siltech® 2145-35 from Siltech Silicones; and Abil T Quat 60^{th} from Evonik.

Some additional non-limiting examples of aminosilicones include the compounds having the following INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22, Quaternium-80, as well as Silicone Quaternium-2 Panthenol Succinate and Silicone Quaternium-16/Glycidyl Dimethicone Crosspolymer.

The one or more silicones can include dimethicones, and/or dimethiconols. The dimethiconols are hydroxyl terminated dimethylsilicones represented by the general chemical formulas (V) and (VI):

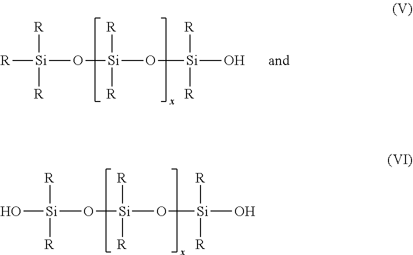

wherein R is an alkyl group (preferably R is methyl or ethyl, more preferably methyl) and x is an integer up to 500, chosen to achieve the desired molecular weight.

Commercial dimethiconol is typically sold as a mixture with dimethicone and/or cyclomethicone (e.g., Dow Coming® 1401, 1402, and 1403 fluids). The silicones in this section may further include any of those disclosed in U.S. Pat. No. 10,828,248.

### Silicone Nanoemulsion

The silicone nanoemulsion comprises nano-sized silicone, non-ionic emulsifier, and carrier fluid. The active percentage of silicone in the emulsion can be 5% to 50%. The hair care composition may include 0.5% to 50% (e.g., 3% to about 30% or even 5% to 25%) of one or more silicone nanoemulsions by weight of the hair care composition. The nano-sized silicone active level contained inside the hair care composition can be 0.1% to 10% (e.g., 0.5% to 6% or 1% to 5%) by weight of the hair care composition.

### Nano-Sized Silicones

The particle size of the one or more nano-sized silicones in the hair care composition can be 1 nm to 100 nm (e.g., 5 nm to 80 nm, 10 nm to 60 nm, 12 nm to 50 nm, or even 20 nm to nm). In some instances, the particle size of the one or more silicones in the hair care composition can be 1 nm to 500 nm (e.g., 5 nm to 300 nm, 8 nm to 200 nm, or even 10 nm to 100 nm).

The particle size of the nanoemulsion silicone can be measured by dynamic light scattering (DLS) using a measurement angle of 173° and the refractive index of the one or more silicones. A Malvern Zetasizer Nano ZEN3600 system using He-Ne laser 633 nm can be used for the measurement at 25° C. For each sample, three particle size measurements are taken and the Z-average value is reported as the particle size.

The one or more silicones may be in the form of a nanoemulsion, i.e., an emulsion in which the particle size is below 100 nm. The nanoemulsion may comprise any silicone suitable for application to the skin and/or hair. From 25% to 99% of the total silicone in the hair care composition may be in the form of a nanoemulsion (e.g., 25% to 75% or 25% to 50%). Silicones described herein may include those disclosed in WO 2019/126442.

For the nano-sized silicone contained in the nanoemulsion, at least one or more silicone may be a polyorganosiloxane compound comprising one or more quaternary ammonium groups, silicone blocks comprising between 80 and 250 siloxane units on average, at least one polyalkylene oxide structural unit, and at least one terminal ester group.

The hair conditioning compositions herein may include a low viscosity silicone polymer that has a viscosity of 100,000 centipoise (cP) or less. For example, the silicone polymer may have a viscosity of 1 cP to 50,000 cP, 25 cP to 30,000 cP, 50 cP to 25,000 cP, 100 cP to 20,000 cP, 150 cP to 15,000 cP, or even 200 cP to 13,000 cP.

Without being bound by theory, the low viscosity silicone polymer provides improved conditioning benefits over conventional silicones because of the addition of hydrophilic functionalities such as, for example, quaternary amines and ethylene oxides/propylene oxides. Compared to silicones with quaternary functionality, these structures are significantly lower in viscosity, so that they don't have to be blended with other lower viscosity diluents and dispersants to allow them to be formulated into products. Low viscosity silicone solvents and diluents can often cause viscosity and stability tradeoffs in hair care products. There is no need for these materials since the silicone polymer is low enough in viscosity to be added directly or in emulsion form. The improved conditioning benefits include smooth feel, reduced friction, and prevention of hair damage, while, in some cases, eliminating the need for a silicone blend.

Structurally, the silicone polymer can be a polyorganosiloxane compound comprising one or more quaternary ammonium groups, at least one silicone block comprising an average between 99 and 199 siloxane units, at least one polyalkylene oxide structural unit, and at least one terminal ester group. The silicone block may comprise an average of 80 to 250 (e.g., 110 to 199, 120 to 199, 130 to 199, 110 to 190, 130 to 190, 110 to 175, 120 to 175, 130 to 175, 110 to 155, 120 to 155, 130 to 155, 155 to 199, 155 to 190, or even 155 to 175) siloxane units. The average block length reflects mean values and can be determined, for example, by 1H-NMR spectroscopy or GPC using protocols known in the art.

The polyorganosiloxane compounds can have a molar ratio of silicone to alkylene oxide block of 2:1 to 20:1 (e.g., 4:1 to about 16:1, 6:1 to 12:1, or even 8:1 to 10:1).

The nitrogen content for the polyorganosiloxane compounds can be 0.1 to 0.4 mmol N/g polymer (e.g., 0.1 to 0.3, 0.13 to 0.27, 0.13 to 0.35, 0.15 to 0.3, 0.17 to 0.27, or even 0.19 to 0.24 mmol N/g polymer).

The polyorganosiloxane compounds herein may have the general formulas (Ia) and (Ib):

M-Y—[—(N⁺R₂-T-N⁺R₂)—Y—]ₘ—[—(NR²-A-E-A′-NR²)—Y-]ₖ-M (Ia)

M-Y—[-—N⁺R₂-T-N⁺R₂)—Y—]ₘ—[—(N⁺R²₂-A-E-A′-N⁺R²₂)—Y-]ₖ-M (Ib)

wherein:
m is >0, alternatively 0.01 to 100, alternatively 0.1 to 100, alternatively 1 to 100, alternatively 1 to 50, alternatively 1 to 20, and alternatively 1 to 10,
k is 0 or an average value of from ≥ 0 to 50, alternatively from ≥ 0 to 20, and alternatively from ≥ 0 to 10,
M represents a terminal group, comprising terminal ester groups selected from

—OC(O)—Z

—OS(O)₂—Z

—OS(O₂)O—Z

—OP(O)(O—Z)OH

—OP(O)(O—Z)₂

wherein Z is selected from monovalent organic residues having up to 40 carbon atoms, optionally comprising one or more hetero atoms;
A and A′ each are independently from each other selected from a single bond or a divalent organic group having up to 10 carbon atoms and one or more hetero atoms, and
E is a polyalkylene oxide group of the general formula:

—[CH₂CH₂O]_{q}—[CH₂CH(CH₃)O]ᵣ—[CH₂CH(C₂H₅)O]ₛ—
wherein q=0 to 200, alternatively 0 to 100, alternatively 0 to 50, alternatively 0 to 25, alternatively 0 to 10, alternatively 1 to 200, alternatively 1 to 100, alternatively 1 to 50, alternatively 1 to 25, and alternatively 1 to 10;
   r=0 to 200, alternatively 0 to 100, alternatively 0 to 50, alternatively 0 to 25, and alternatively 0 to 10;
   s=0 to 200, alternatively 0 to 100, alternatively 0 to 50, alternatively 0 to 25, and alternatively 0 to 30;
   q+r+s=1 to 600, or alternatively from 1 to 100, or alternatively from 1 to 50, or alternatively from 1 to 40
   with percentage of q in (q/(q+r+s))=0%, 0.166% to 100%, 1% to 100%, 2% to 100%, 2.5% to 100%, 10% to 100%, 30% to 100%, 50% to 100%; alternatively percentage of q in (q/(q+r+s))=at least 1%, alternatively at least 2%, alternatively at least 10%, alternatively at least 30% alternatively at least 50%, alternatively at least 75%, alternatively at least 90%, alternatively at least 95%, and alternatively 100%.
R² is selected from hydrogen or R,
R is selected from monovalent organic groups having up to 22 carbon atoms and optionally one or more heteroatoms, and wherein the free valencies at the nitrogen atoms are bound to carbon atoms,
Y is a group of the formula:

-K-S-K-and -A-E-A′- or -A′-E-A-,

with

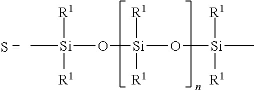

wherein R1=C₁-C₂₂-alkyl, C₁-C₂₂-fluoralkyl or aryl; n=99 to 199 on average, alternatively 110 to 199 on average, alternatively 120 to 199 on average, alternatively 130 to 199 on average, alternatively 110 to 190 on average, alternatively 130 to 190 on average, alternatively 110 to 175 on average, alternatively 120 to 175 on average, alternatively 130 to 175 on average, alternatively 110 to 155 on average, alternatively 120 to 155 on average, alternatively 130 to 155 on average, alternatively 155 to 199 on average, alternatively 155 to 190 on average, alternatively 155 to 175 on average and these can be identical or different if several S Groups are present in the polyorganosiloxane compound;
   K is a bivalent or trivalent straight chain, cyclic and/or branched C₂-C₄₀ hydrocarbon residue which is optionally interrupted by —O—, —NH—, trivalent N, —NR¹—, —C(O)—, —C(S)—, and optionally substituted with —OH, wherein R¹ is defined as above,
T is selected from a divalent organic group having up to 20 carbon atoms and optionally one or more hetero atoms.

The residues K may be identical or different from each other. In the -K-S-K-moiety, the residue K is bound to the silicon atom of the residue S via a C—Si-bond.

Due to the possible presence of amine groups (—(NR²-A-E-A′-NR²)—) in the polyorganosiloxane compounds, they may have protonated ammonium groups, resulting from the protonation of such amine groups with organic or inorganic acids. Such compounds are sometimes referred to as acid addition salts of the polyorganosiloxane compounds according to the invention.

The molar ratio of the quaternary ammonium groups b) and the terminal ester groups c) is less than 20:3, alternatively less than 5:1, alternatively less than 10:3 and alternatively less than 2:1. The ratio can be determined by ¹³C-NMR or 1H-NMR.

The silicone polymer has a viscosity at 20° C.and a shear rate of 0.1 s⁻¹ (plate-plate system, plate diameter 40 mm, gap width 0.5 mm) of less than 100,000 mPa·s. For example, the viscosities of the neat silicone polymers may range from 500 to 100,000 mPa·s (e.g., 500 to 70,000 mPa·s, 500 to 50,000 mPa·s, 500 to 30,000 mPa·s, 2,000 to 100,000 mPa·s, 2,000 to 70,000 mPa·s, 2,000 to 50,000 mPa·s, 2,000 to 30,000 mPa·s, 8,000 to 100,000 mPa·s, 8,000 to 70,000 mPa·s, 8,000 to 50,000 mPa·s, 8,000 to 30,000 mPa·s, 15,000 to 100,000 mPa·s, 15,000 to 70,000 mPa·s, 15,000 to 50,000 mPa·s, or even 15,000 to 30,000 mPa·s) determined at 20° C.and a shear rate of 0.1 s⁻¹.

In addition to the above listed silicone polymers, it can be desirably to use the polymers provided below. For example, in the polyalkylene oxide group E of the general formula:

—[CH₂CH₂O]_{q}—[CH₂CH(CH₃)O]ᵣ—[CH₂CH(C₂H₅)O]ₛ—

wherein the q, r, and s indices may be defined as follows:
q=1 to 200, or preferably from 1 to 100, or more preferably from 1 to 50, or even more preferably from 1 to 20,
r=0 to 200, or preferably from 0 to 100, or more preferably from 0 to 50, or even more preferably from 0 to 20,
s=0 to 200, or preferably from 0 to 100, or more preferably from 0 to 50, or even more preferably from 0 to 20,
q+r+s=1 to 600, or preferably from 1 to 100, or more preferably from 1 to 50, or even more preferably from 1 to 40
with percentage of q in (q/(q+r+s)) 0%, 0.166% to 100%, 1% to 100%, 2% to 100%, 2.5% to 100%, 10% to 100%, 30% to 100%, 50% to 100%.

For polyorganosiloxane structural units with the general formula S:

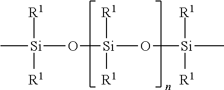

R¹=C₁-C₂₂-alkyl, C₁-C₂₂-fluoralkyl or aryl; n=from 99 to 199, K (in the group -K-S-K-) is preferably a bivalent or trivalent straight chain, cyclical or branched C₂-C₂₀ hydrocarbon residue which is optionally interrupted by —O—, —NH—, trivalent N, —NR¹—, —C(O)—, —C(S)—, and optionally substituted with —OH.

R¹ can be C₁-C₁₈ alkyl, C₁-C₁₈ fluoroalkyl and aryl. Furthermore, R¹ is preferably C₁-C₁₈ alkyl, C₁-C₆ fluoroalkyl and aryl. Furthermore, R¹ is more preferably C₁-C₆ alkyl, C₁-C₆ fluoroalkyl, even more preferably C₁-C₄ fluoroalkyl, and phenyl. Most preferably, R¹ is methyl, ethyl, trifluoropropyl and phenyl.

As used herein, the term “C₁-C₂₂ alkyl” means that the aliphatic hydrocarbon groups possess from 1 to 22 carbon atoms which can be straight chain or branched. Methyl, ethyl, propyl, n-butyl, pentyl, hexyl, heptyl, nonyl, decyl, undecyl, isopropyl, neopentyl and 1,2,3-trimethyl hexyl moieties serve as examples. As used herein, the term “C₁-C₂₂ fluoroalkyl” means aliphatic hydrocarbon compounds with 1 to 22 carbon atoms which can be straight chain or branched and are substituted with at least one fluorine atom. Some non-limiting examples are monofluormethyl, monofluoroethyl, 1,1,1-trifluorethyl, perfluoroethyl, 1,1,1-trifluoropropyl and 1,2,2-trifluorobutyl. The term “aryl” includes unsubstituted or phenyl substituted one or more times with OH, F, Cl, CF₃, C₁-C₆ alkyl, C₁-C₆ alkoxy, C₃-C₇ cycloalkyl, C₂-C₆ alkenyl or phenyl. Aryl also includes naphthyl.

The positive charges resulting from the ammonium group(s) of the polyorganosiloxane, can be neutralized with inorganic anions such as chloride, bromide, hydrogen sulfate, sulfate, or organic anions, like carboxylates deriving from C₁-C₃₀ carboxylic acids, for example acetate, propionate, octanoate, especially from C₁₀-C₁₈ carboxylic acids, for example decanoate, dodecanoate, tetradecanoate, hexadecanoate, octadecanoate and oleate, alkylpolyethercarboxylate, alkylsulphonate, arylsulphonate, alkylarylsulphonate, alkylsulphate, alkylpolyethersulphate, phosphates derived from phosphoric acid mono alkyl/aryl ester and phosphoric acid dialkyl/aryl ester. The properties of the polyorganosiloxane compounds can be, inter alia, modified based upon the selection of acids used.

The quaternary ammonium groups are usually generated by reacting the di-tertiary amines with an alkylating agents, selected from in particular di-epoxides (sometimes referred to also as bis-epoxides) in the presence of mono carboxylic acids and difunctional dihalogen alkyl compounds.

The polyorganosiloxane compounds can be of the general formulas (Ia) and (Ib):

M-Y—[—(N⁺R₂-T-N⁺R₂)—Y—]ₘ—[—(NR²-A-E-A′-NR²)—Y-]ₖ-M (Ia)

M-Y—[—(N⁺R₂-T-N⁺R₂)—Y—]ₘ—[—(N⁺R²₂1A-E-A′-N⁺R²₂)—Y-]ₖ-M (Ib)

wherein each group is as defined above; however, the repeating units are in a statistical arrangement (i.e., not a block-wise arrangement).

The polyorganosiloxane compounds may be also of the general formulas (IIa) or (IIb):

M-Y—[—N⁺R₂—Y—]ₘ—[—(NR²-A-E-A′-NR²)—Y-]ₖ-M (IIa)

M-Y—[—N⁺R₂—Y—]ₘ—[—(N⁺R²₂-A-E-A′-N⁺R²₂)—Y-]ₖ-M (IIb)

wherein each group is as defined above. Also in such formula the repeating units are usually in a statistical arrangement (i.e not a block-wise arrangement).
wherein, as defined above, M is

—OC(O)—Z,

—OS(O)₂—Z

—OS(O₂)O—Z

—OP(O)(O—Z)OH

OP(O)(O—Z)₂

Z is a straight chain, cyclic or branched saturated or unsaturated C₁-C₂₀, or preferably C₂ to C₁₈, or even more preferably a hydrocarbon radical, which can be interrupted by one or more —O—, or —C(O)— and substituted with —OH. M can be —OC(O)—Z resulting from normal carboxylic acids in particular with more than 10 carbon atoms like for example dodecanoic acid.

The molar ratio of the polyorganosiloxane-containing repeating group -K-S-K- and the polyalkylene repeating group -A-E-A′- or -A′-E-A- is between 1:100 and 100:1, alternatively between 1:20 to 20:1, or alternatively between 1:10 to 10:1.

In the group —(N⁺R₂-T-N⁺R₂)—, R may represent a monovalent straight chain, cyclic or branched C₁-C₂₀ hydrocarbon radical, which can be interrupted by one or more —O—, —C(O)— and can be substituted by —OH, T may represent a divalent straight-chain, cyclic, or branched C₁-C₂₀ hydrocarbon radical, which can be interrupted by —O—, —C(O)— and can be substituted by hydroxyl.

The above described polyorganosiloxane compounds comprising quaternary ammonium functions and ester functions may also contain: 1) individual molecules which contain quaternary ammonium functions and no ester functions; 2) molecules which contain quaternary ammonium functions and ester functions; and 3) molecules which contain ester functions and no quaternary ammonium functions. While not limited to structure, the above described polyorganosiloxane compounds comprising quaternary ammonium functions and ester functions are to be understood as mixtures of molecules comprising a certain averaged amount and ratio of both moieties. A particularly suitable polyorganosiloxane comprising quaternary ammonium functions is the product having the commercial name Momentive Waro® Y20875, Silsoft® Q (emulsion).

The composition may be free of amodimethicone.

Various monofunctional organic acids may be utilized to yield the esters and include C₁-C₃₀ carboxylic acids (e.g., C₂, C₃, C₈, C₁₀, C₁₂, C₁₄, C₁₆ or even C₁₈ carboxylic acids), saturated, unsaturated and hydroxyl functionalized C18 acids, alkylpolyethercarboxylic acids, alkylsulphonic acids, arylsulphonic acids, alkylarylsulphonic acids, alkylsulphuric acids, alkylpolyethersulphuric acids, phosphoric acid mono alkyl/aryl esters and phosphoric acid dialkyl/aryl esters.

### Nonionic Emulsifiers

The hair conditioning compositions herein may include 0% to 20% (e.g., 0.01% to 19%, 0.05% to 18%, 0.1% to 17%, 1% to 15%, 2% to 10% or even 2.5% to 7.5%) of a nonionic emulsifier, by weight of the hair care composition. Nonionic emulsifiers may be broadly defined as including compounds containing an alkylene oxide groups (hydrophilic in nature) with a hydrophobic compound, which may be aliphatic or alkyl aromatic in nature. Examples of nonionic emulsifiers include alcohol ethoxylates, polyethylene oxide condensates of alkyl phenols, condensation products of ethylene oxide and the product resulting from the reaction of propylene oxide and ethylene diamine products, long chain tertiary amine oxides, long chain tertiary phosphine oxides, long chain dialkyl sulfoxides containing one short chain alkyl or hydroxy alkyl radical of 1 to 3 carbon atoms (usually methyl) and one long hydrophobic chain which includes alkyl, alkenyl, hydroxy alkyl, or keto alkyl radicals containing 8 to 20 carbon atoms, 0 to 10 ethylene oxide moieties and 0 or 1 glyceryl moiety, polysorbates (e.g., sucrose esters of fatty acids), alkyl polysaccharide nonionic emulsifiers (e.g., as disclosed in U.S. Pat. No. 4,565,647), and polyethylene glycol (PEG) glyceryl fatty esters.

Particularly suitable non-ionic emulsifiers can be alcohol ethoxylates that are condensation products of C₈₋₁₈ aliphatic alcohols in either straight chain or branched chain configuration, with 2 to 35 moles of ethylene oxide (e.g., a coconut alcohol ethylene oxide condensate with 2 to 30 moles of ethylene oxide per mole of coconut alcohol, wherein the coconut alcohol fraction has 10 to 14 carbon atoms and condensation products of alkyl phenols with an alkyl group containing 6 to 20 carbon atoms in either a straight chain or branched chain configuration, with ethylene oxide, wherein the ethylene oxide is present at 3 to 60 moles of ethylene oxide per mole of alkyl phenol.

In some aspects, the nonionic emulsifier may be a silicone emulsifier. A wide variety of silicone emulsifiers may be useful herein. These silicone emulsifiers are typically organically modified siloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

The nanoemulsions herein may be prepared by mechanical and/or emulsion polymerization. Mechanical polymerization techniques involve: (1) dissolving a primary surfactant in water, (2) adding a silicone to form a two-phase mixture, (3) slowly adding a co-surfactant to the 2-phase mixture while mixing until a clear isotropic microemulsion of a siloxane-in-water is formed. Emulsion polymerization techniques involve adding: (1) a polymer precursor(s) (i.e., monomers, or reactive oligomers, which are immiscible in water), (2) a surfactant to stabilize polymer precursor droplets in water, and (3) a water-soluble polymerization catalyst (e.g., a strong mineral acid such as hydrochloric acid, or a strong alkaline catalyst such as sodium hydroxide) to water and stirring. Polymerization is allowed to advance until the reaction is complete, or the desired degree of polymerization (DP) is reached, and an emulsion of the polymer is formed.

Some additional non-limiting examples of emulsifiers that may be suitable for use herein are disclosed in WO 2019/126442.

### Solvent/Carrier Fluid

Inclusion of an appropriate quantity of a carrier fluid can facilitate the formation of a silicone emulsion and suspend the nano-sized silicone maintaining its size stability for an extended period of time. A silicone nanoemulsion composition can include, by weight of the composition, 50% to 95%, of a liquid carrier (e.g., 60% to 85% or 65% to 80%).

A liquid carrier can be water or can be a miscible mixture of water and organic solvent. A liquid carrier can be water with minimal or no significant concentrations of organic solvent, except as otherwise incidentally incorporated into the composition as minor ingredients of other essential or optional components. Suitable organic solvents can include water solutions of lower alkyl alcohols and polyhydric alcohols. Useful lower alkyl alcohols include monohydric alcohols having 1 to 6 carbons, such as ethanol and isopropanol. Exemplary polyhydric alcohols include propylene glycol, hexylene glycol, glycerin, and propane diol.

### High Melting Point Fatty Compounds

The hair care composition may include 0.5% to 15% (e.g., 1% to 13% or 2% to 10%) high melting point fatty compounds, by weight of the hair care composition. The high melting point fatty compounds have a melting point of about 25° C. or higher (e.g., 45° C., 60° C.or higher) and can be selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives.

However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The fatty alcohols described herein are those with 14 to 30 (e.g., 16 to 22) carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having 10 to 30 carbon atoms (e.g., 12 to 22 carbon atoms or 16 to about 20 carbon atoms). These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy- substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through steareth-10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e., a mixture of fatty alcohols containing predominantly cetyl and stearyl alcohol, wherein the numeric designation indicates the number of ethylene glycol moieties present; C16 -C30 alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

The fatty compound may be a single high melting point compound of high purity. Single compounds of pure fatty alcohols selected may be selected from the group consisting of pure cetyl alcohol, stearyl alcohol, and behenyl alcohol. By “pure” herein, what is meant is that the compound has a purity of at least 90%, alternatively at least 95%.

### Cationic Surfactants

The hair care composition described herein may include 0% to 10% of one or more cationic surfactants (e.g., 0.25% to 9%, 0.5% to 7.5%, 1% to about 6%, 2% to 5%, or 3% to 6%) by weight of the hair care composition.

The cationic surfactant can be selected from the group consisting of mono-long alkyl quaternized ammonium salts, di-long alkyl quaternized ammonium salts, mono-long alkyl amidoamine salts, and mixtures thereof.

### (i) Mono-Long Alkyl Quaternized Ammonium Salts

The cationic surfactant can be a mono-long alkyl quaternized ammonium salt having the formula (VII), e.g., as disclosed in WO2013148778:

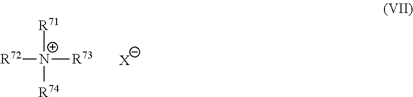

wherein one of R⁷¹, R⁷² R⁷³ a n R⁷⁴ selected from an aliphatic group of from about 14 to about 30 carbon atoms or an aromatic, alkoxy, polyoxyalkylene, alkylamido, hydroxyalkyl, aryl or alkylaryl group having up to about 30 carbon atoms; the remainder of R⁷¹, R⁷² R⁷³ and R⁷⁴ are independently selected from an aliphatic group of from about 1 to about 8 carbon atoms or an aromatic, alkoxy, polyoxyalkylene, alkylamido, hydroxyalkyl, aryl or alkylaryl group having up to about 8 carbon atoms; and X is a salt-forming anion such as those selected from halogen, (e.g., chloride, bromide), acetate, citrate, lactate, glycolate, phosphate, nitrate, sulfonate, sulfate, alkylsulfate, glutamate, and alkyl sulfonate radicals. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, one of R⁷¹, R⁷², R⁷³ and R⁷⁴ is selected from an alkyl group of from about 14 to about 30 carbon atoms, more preferably from about 16 to about 22 carbon atoms, still more preferably from about 16 to about 18 carbon atoms; the remainder of R⁷¹, R⁷², R⁷³, and R⁷⁴ are independently selected from the group consisting of CH₃, C₂H₅, C₂H₄OH, CH₂C₅H₅, and mixtures thereof; and (X) is selected from the group consisting of Cl, Br, CH₃OSO₃, and mixtures thereof. Mono-long alkyl quatemized ammonium salts can provide improved slippery and slick feel on wet hair.

Nonlimiting examples of such mono-long alkyl quatemized ammonium salt cationic surfactants include: behenyl trimethyl ammonium chloride available, for example, with tradename Genamine KDMP from Clariant, with tradename INCROQUAT TMC-80 from Croda and ECONOL TM22 from Sanyo Kasei; stearyl trimethyl ammonium chloride available, for example, with tradename CA-2450 from Nikko Chemicals; cetyl trimethyl ammonium chloride available, for example, with tradename CA-2350 from Nikko Chemicals; behenyltrimethylammonium methyl sulfate, available from FeiXiang; hydrogenated tallow alkyl trimethyl ammonium chloride; stearyl dimethyl benzyl ammonium chloride; and stearoyl amidopropyl dimethyl benzyl ammonium chloride.

### (ii) Mono-Long Alkyl Amidoamine Salts

Mono-long alkyl amines can also be suitable as cationic surfactants. Primary, secondary, and tertiary fatty amines can be useful. The cationic surfactants can be tertiary amido amines having an alkyl group of 12 to 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Additional cationic surfactant amines are disclosed in U.S. Pat. No. 4,275,055.

These amines can also be used in combination with acids such as l-glutamic acid, lactic acid, hydrochloric acid, malic acid, succinic acid, acetic acid, fumaric acid, tartaric acid, citric acid, (-glutamic hydrochloride, maleic acid, and mixtures thereof; more preferably l-glutamic acid, lactic acid, citric acid. The amines herein can be partially neutralized with any of the acids at a molar ratio of the amine to the acid of 1:0.3 to 1:2 or 1:0.4 to 1:1.

(iii) Di-long alkyl quaternized ammonium salts

The cationic surfactants described herein can be di-long alkyl quaternized ammonium salts. Di-long alkyl quaternized ammonium salts can be combined with a mono-long alkyl quaternized ammonium salt or mono-long alkyl amidoamine salt. Such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts can be used at a level such that the wt% of the dialkyl quaternized ammonium salt in the cationic surfactant system is in the range of 10% to 50% or 30% to 45%.

Di-alkyl cationic surfactants useful herein can be those having two long alkyl chains of 12 to 30 carbon atoms (e.g., 16 to 24 carbon atoms or 16 to 22 carbon atoms) including, for example, di-long alkyl quaternized ammonium salts. Di-alkyl quaternized ammonium salts that may be useful herein are those having the formula (VIII):

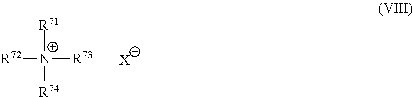

wherein two of R⁷¹, R⁷², R⁷³ and R⁷⁴ are selected from an aliphatic group of 12 to 30 carbon atoms, (e.g., 16 to 24 carbon atoms or 16 to 22 carbon atoms) or an aromatic, alkoxy, polyoxyalkylene, alkylamido, hydroxyalkyl, aryl or alkylaryl group having up to 30 carbon atoms; the remainder of R⁷¹, R⁷², R⁷³ and R⁷⁴ are independently selected from an aliphatic group of 1 to 8 carbon atoms, preferably 1 to 3 carbon atoms, or an aromatic, alkoxy, polyoxyalkylene, alkylamido, hydroxyalkyl, aryl or alkylaryl group having up to 8 carbon atoms; and X⁻ is a salt-forming anion selected from halides such as chloride and bromide, C1-C4 alkyl sulfate such as methosulfate and ethosulfate, and mixtures thereof. The aliphatic groups may contain, in addition to carbon and hydrogen atoms, ether linkages, and other groups such as amino groups. The longer chain aliphatic groups, e.g., those of about 16 carbons or higher, can be saturated or unsaturated. Two of R⁷¹, R⁷², R⁷³ and R⁷⁴ can be selected from an alkyl group of from 12 to 30 carbon atoms (e.g., 16 to 24 carbon atoms or 18 to 22 carbon atoms); and the remainder of R⁷¹, R⁷², R⁷³ and R⁷⁴ can be independently selected from CH₃, C₂H₅, C₂H₄OH, CH₂C₆H₅, and mixtures thereof.

Additional di-alkyl cationic surfactants can include dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### Water Miscible Solvents

The hair care composition described herein can include 0.1% to 15% (e.g., 0.2% to 10% or 0.3% to about 5%) of a water miscible solvent, by weight of the hair care composition. Some non- limiting examples of water miscible solvents that may be suitable for use herein include polyols, copolyols, polycarboxylic acids, polyesters, and alcohols.

Some non-limiting examples polyols are glycerin, diglycerin, propylene glycol, ethylene glycol, butylene glycol, pentylene glycol, 1,3-butylene glycol, cyclohexane dimethanol, hexane diol, polyethylene glycol (200-600), sugar alcohols such as sorbitol, manitol, lactitol and other mono- and polyhydric low molecular weight alcohols (e.g., C₂-C₈ alcohols); mono di- and oligo-saccharides such as fructose, glucose, sucrose, maltose, lactose, and high fructose corn syrup solids and ascorbic acid.

Some non-limiting examples of polycarboxylic acids are citric acid, maleic acid, succinic acid, polyacrylic acid, and polymaleic acid.

Some non-limiting examples of polyesters are glycerol triacetate, acetylated- monoglyceride, diethyl phthalate, triethyl citrate, tributyl citrate, acetyl triethyl citrate, acetyl tributyl citrate.

Some non-limiting examples of dimethicone copolyols are PEG-12 dimethicone, PEG/PPG-18/18 dimethicone, and PPG-12 dimethicone.

Some non-limiting examples of alcohols are ethanol, n-propanol, isopropanol, n-butanol, sec-butanol, tert-butanol, n-hexanol and cyclohexanol.

The water miscible solvents may be selected from glycerin, propylene glycol, dipropylene glycol, and mixtures thereof. EP 0283165 B1 discloses other suitable water miscible solvents, including glycerol derivatives such as propoxylated glycerol. In some aspect, glycerin may be particularly suitable.

### Viscosity Modifiers

The hair care composition described herein can include 0.1% to 2% (e.g., 0.1% to 1% or 0.1% to 0.5%) of a viscosity modifier, by weight of the hair care composition. Some non-limiting examples of viscosity modifiers that may be suitable for use herein include water soluble polymers and cationic water soluble polymers.

Some non-limiting examples of water soluble polymers are (1) vegetable based polymers such as gum Arabic, tragacanth gum, galactan, guar gum, carob gum, karaya gum, carrageenan, pectin, agar, quince seed, algal colloid, starch (rice, corn, potato, or wheat), and glycyrrhizinic acid; (2) microorganism-based polymers such as xanthan gum, dextran, succinoglucan, and pullulan; and (3) animal-based polymers such as collagen, casein, albumin, and gelatin. Examples of semi- synthetic water-soluble polymers include (1) starch-based polymers such as carboxymethyl starch and methylhydroxypropyl starch; (2) cellulose-based polymers such as methylcellulose, nitrocellulose, ethylcellulose, methylhydroxypropylcellulose, hydroxyethylcellulose, sodium cellulose sulfate, hydroxypropylcellulose, sodium carboxymethylcellulose (CMC), crystalline cellulose, and cellulose powder; and (3) alginate-based polymers such as sodium alginate and propylene glycol alginate. Examples of synthetic water-soluble polymers include (1) vinyl-based polymers such as polyvinyl alcohol, polyvinyl methyl ether- based polymer, polyvinylpyrrolidone, and carboxyvinyl polymer (CARBOPOL 940, CARBOPOL 941; (2) polyoxyethylene-based polymers such as polyethylene glycol 20,000, polyethylene glycol 6,000, and polyethylene glycol 4,000; (3) copolymer-based polymers such as a copolymer of polyoxyethylene and polyoxypropylene, and PEG/PPG methyl ether; (4) acryl-based polymers such as poly(sodium acrylate), poly(ethyl acrylate), polyacrylamide, polyethylene imines, and cationic polymers. The water-swellable clay minerals are nonionic water-soluble polymers and correspond to one type of colloid-containing aluminum silicate having a triple layer structure. More particular, as examples thereof, mention may be made of bentonite, montmorillonite, beidellite, nontronite, saponite, hectorite, aluminum magnesium silicate, and silicic anhydride.

Some non-limiting examples of cationic water soluble polymers are (1) quaternary nitrogen-modified polysaccharides such as cation-modified cellulose, cation-modified hydroxyethylcellulose, cation-modified guar gum, cation-modified locust bean gum, and cation-modified starch; (2) dimethyldiallylammonium chloride derivatives such as a copolymer of dimethyldiallylammonium chloride and acrylamide, and poly(dimethylmethylene piperidinium chloride); (3) vinylpyrrolidone derivatives such as a salt of a copolymer of vinylpyrrolidone and dimethylaminoethyl methacrylic acid, a copolymer of vinylpyrrolidone and methacrylamide propyltrimethylammonium chloride, and a copolymer of vinylpyrrolidone and methylvinylimidazolium chloride; and (4) methacrylic acid derivatives such as a copolymer of methacryloylethyldimethylbetaine, methacryloylethyl trimethylammonium chloride and 2-hydroxyethyl methacrylate, a copolymer of methacryloylethyldimethylbetaine, and methacryloylethyl trimethylammonium chloride and methoxy polyethylene glycol methacrylate.

### Rheology

The hair care composition described herein can have a shear stress of 10 Pa to 800 Pa (e.g., 20 Pa to 600 Pa, 40 Pa to 500 Pa or 50 Pa to 400 Pa) at a shear rate of 950 s⁻¹.

The liquid phase rheology values of the hair care composition described herein can be measured employing any suitable rheometer or viscometer at 26.7° C.and at a shear rate ramp from 0 to 1100 s⁻¹ and taking the reading at 950 s⁻¹.

For example, the liquid phase rheology values reported in the data herein were measured using a Discovery HR-2 rheometer, by TA Instruments Inc. The cone used (cone H/A-AL ST 40 MM 2DEG Smart-Swab) has a diameter of 40 mm and 2° angle with designated gap by the specific cone (typically 50 μm). Shear rate is logarithmically increased from 0.1 to 1100 s⁻¹ in 1 min, and temperature is kept at 26.7° C. The shear stress at shear rate=950 s⁻¹ is read. The sample size was 2.5 ml.

### Optional Ingredients

The hair care composition described herein can optionally comprise one or more additional components known for use in hair care or personal care products, provided that the additional components are physically and chemically compatible with the essential components described herein, or do not otherwise unduly impair product stability, aesthetics or performance. Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the conditioning composition.

Some non-limiting examples of optional ingredients include preservatives, perfumes or fragrances, cationic polymers, viscosity modifiers, coloring agents or dyes, conditioning agents, hair bleaching agents, thickeners, moisturizers, foam boosters, additional surfactants or nonionic cosurfactants, emollients, pharmaceutical actives, vitamins or nutrients, sunscreens, deodorants, sensates, plant extracts, nutrients, astringents, cosmetic particles, absorbent particles, adhesive particles, hair fixatives, fibers, reactive agents, skin lightening agents, skin tanning agents, anti-dandruff agents, perfumes, exfoliating agents, acids, bases, humectants, enzymes, suspending agents, pH modifiers, hair colorants, hair perming agents, pigment particles, anti-acne agents, anti-microbial agents, sunscreens, tanning agents, exfoliation particles, hair growth or restorer agents, insect repellents, shaving lotion agents, non-volatile solvents or diluents (water-soluble and water-insoluble), co-solvents or other additional solvents, and similar other materials.

### Water

The hair care composition described herein may include 55% to 90% water (e.g., 65% to 87.5%, 67.5% to 85%, 70% to 82.5% or even 72.5% to 80%), by weight of the hair care composition.

### Method of Conditioning the Hair

The method of conditioning the hair described herein comprises (1) providing a hair care composition as described herein; (2) applying the composition to the hair; and (3) rinsing the composition from the hair. Hair switches were treated in above method too for testings performed below.

### EXAMPLES

The following examples illustrate the hair care composition and/or method of conditioning the hair described herein. The exemplified compositions can be prepared by conventional formulation and mixing techniques, for example as described herein. Some components may come from suppliers as dilute solutions. The amount stated reflects the weight percent of the active material, unless otherwise specified.

The hair conditioning compositions herein do not accumulate on the hair over time and do not weigh the hair down, and surprisingly maintain a good dry conditioning benefit. It is believed both silicone particle size, its emulsifying system, and also the silicone type give rise to this difference in deposition. In the beginning, the silicone particle size and its emulsifying system determine how homogeneous silicone deposits for one-cycle usage. But over time, the silicone property, for example ADM8100E (CC05 amodimethicone) being a durable silicone, is very hard to be washed out and accumulates over multi-cycle usage. Normally, conditioners with neat silicone also have this accumulation over multi-cycle usage. In contrast, a polyquaternium silicone nanoemulsion, for example Momentive Waro Y20875, after blending with a neat silicone, doesn't have this tight deposition property and can be washed out every time.

The following conditioner compositions in Table 1 were prepared by creating a conditioner base with fatty alcohol, cationic surfactant, water, EDTA and benzyl alcohol, and post-adding the silicone or blends and perfume by centrifugal mixing. The conditioner base is made by heating up the fatty alcohol and cationic surfactant mixture, and inline mix with water phase at high shear to form the conditioner gel network structure. The post-addition of silicone and perfume was done by a FlackTek SpeedMixer® at a mixing speed of 2000 rpm for two minutes.

TABLE 1
Hair Conditioner Composition

| | Inventive | Inventive | Inventive | Comparative | Comparative | Comparative |
|---|---|---|---|---|---|---|
| Ingredients | Example 1 | Example 2 | Example 3 | Example 4 | Example 5 | Example 6 (ADM) |
| ADM8100E (20% Active) | | | | | | 10 |
| Waro Y20875 (20% Active) | 10 | 15 | 5 | 0 | 20 | |
| Terminal Modified Amino Silicone | 2 | 1 | 3 | 4 | 0 | 2 |
| Fluid* (neat silicone) | | | | | | |
| Behentrimonium Methosulfate | 3.92 | 3.92 | 3.92 | 3.92 | 3.92 | 3.92 |
| (MW = 479.8 g/mol) (80% Active**) | | | | | | |
| Cetyl Alcohol (MW = 242.44 g/mol) | 1.5561 | 1.5561 | 1.5561 | 1.5561 | 1.5561 | 1.5561 |
| Stearyl Alcohol (MW = 270.49 g/mol) | 3.891 | 3.891 | 3.891 | 3.891 | 3.891 | 3.891 |
| Methylchloroisothiazonlinone/ | 0.00045 | 0.00045 | 0.00045 | 0.00045 | 0.00045 | 0.00045 |
| Methylisothiazonlinone | | | | | | |
| Benzyl alcohol | 0.4 | 0.4 | 0.4 | 0.4 | 0.4 | 0.4 |
| Ethylenediaminetetraacetic acid | 0.127 | 0.127 | 0.127 | 0.127 | 0.127 | 0.127 |
| Perfume | 0.75 | 0.75 | 0.75 | 0.75 | 0.75 | 0.75 |
| Water | Balance | Balance | Balance | Balance | Balance | Balance |
| Rheology (Shear Stress at 950 s⁻¹ Shear Rate) | 206.515 Pa | 160.04 Pa | 126.08 Pa | 373.53 Pa | 162.227 Pa | 146 Pa |

*Y-14945 from Momentive
**BTMS P8580KC from KCI Limited.

### Methods

### IR Micro Imaging (Transmission Mode)

FTIR microscopy is done on a conditioner composition to visually assess distribution of silicones. Samples are manually coated using cleaned microscope glass on 2×2 cm CaF₂ IR transparent crystal to form thin film/coating and subsequently dried in the air for a few hours prior to measurement.

The measurement is done using Bruker Vertex 80v FTIR with Hyperion 3000 equipped with 128×128 Focal plane Array Detector (FPA) in transmission mode with 36×IR Objective lens and 15×IR condenser lens from 4000-900 cm⁻¹, minimum of 1024 scans and resolution: 8 cm⁻¹. The background is collected on empty CaF₂ crystal prior sample measurement. For each sample, the measurement is done 2×2 adjoining single images of the sample (tiles) on 3 replicates from different random area on the CaF₂ substrate.

Initial spectrum processing is done in Bruker OPUS Software version 6.0 or higher, where the spectra is cut from 3800-950 cm^{-1,} straight line generation from 1900-2500 cm⁻¹ and baseline correction using rubber band method. Further analysis is done in Malvern ISys version 5.0 to concatenate 3 replicates of 2×2 adjoining single images in x-direction and different samples in y-direction. The silicone distribution is calculated using peak ratio between Si—CH₃ at 1259 cm⁻¹ to CH₂ at 2921 cm⁻¹.

### Instron Friction Method (IFM)

IFM (Instron Friction Method TMD01645 IFM-1) is used to determine the surface friction on dry hair switches after treatment with shampoo/rinse off conditioner/leave on treatment (LOT). The purpose is to evaluate hair surface smoothness. The lower friction value indicates more smoothness on the hair surface. A value lower than 70 is considered comparatively smooth.

Pendulum Free Flow Method Pendulum (TMD01646 Pendulum Free Flow Method II) is used to determine the force exerted by swinging dry hair switches after treatment with shampoo/rinse off conditioner/LOT. The purpose is to evaluate hair free flow in pendulum motion. Higher measurement results describe better hair free flowing benefit. A value higher than 1 is considered relatively free-flowing.

### Silicone Deposition

Total silicone deposited on hair is extracted using organic solvent, hexane: isopropyl alcohol (1:1 ratio). Extracted silicone from hair is measured using Inductively Coupled Plasma - Optical Emission Spectroscopy (ICP-OES). This technique will measure silicon as an element contained in silicone polymer. Quantitative analysis of Si by ICP-AES is done at 251.432 nm.

### Fatty Alcohol Deposition

Cetyl alcohol and stearyl alcohol on hair are extracted from hair switches with hexane:isopropyl alcohol (1:1 ratio) containing 1-nonadecanol as an internal standard. Sample extract is then injected into the Gas Chromatography with Flame-Ionization detection (FID) for quantification and the amount of cetyl alcohol and stearyl alcohol is summed up and calculated as Total Fatty Alcohol.

### Cationic Surfactants Deposition

Cationic surfactants like behentrimonium methosulfate (BTMS) are extracted from hair switches with hexane:isopropyl alcohol (1:1 ratio) and analyzed using high-performance liquid chromatography (HPLC) with a strong cation exchange column and then detected by Evaporative light scattering detector (ELSD).

### Scanning Electron Microscopy (SEM)

SEM micrographs were obtained with Zeiss Crossbeam 540 Scanning electron microscope operating at 10 kV acceleration voltage using secondary electron detectors and an energy-dispersive X-ray microanalysis system (Bruker). A small piece (1 cm in length) of hair strand was cut from the middle of the hair switch and mounted on a copper sample holder using conductive double sided adhesive carbon tape, and then sputter-coated with a thin layer of platinum-palladium in Quorum PP3010 preparation chamber at room temperature to guarantee their electrical conductivity. Five strands were analyzed per hair switch at 2000× and 5000× magnification for surface morphology as well as Si elemental map to reflect silicone distribution.

### Testing Results

### IR Micro Imaging Results

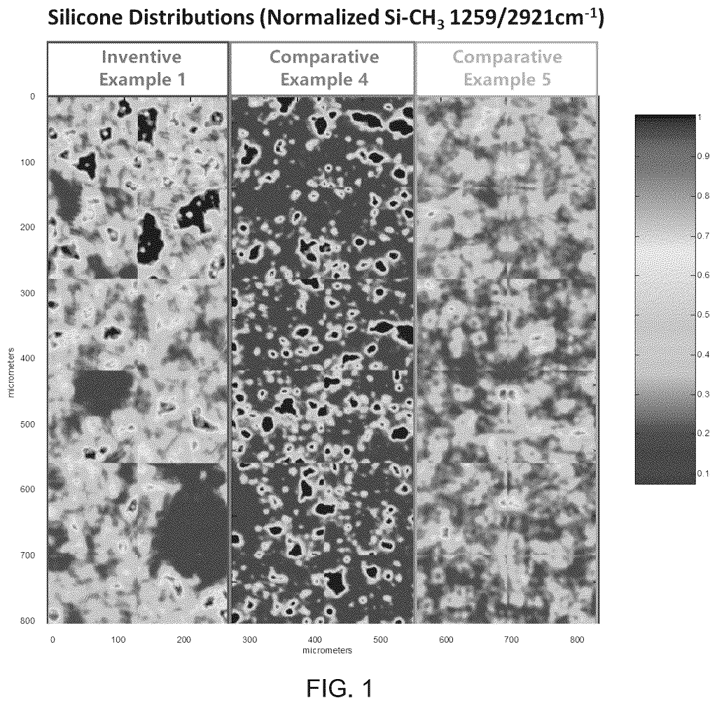

FIG. 1 shows the microscopy of silicone distribution of three conditioners. The microscope image for conditioners with only neat silicone has distinct silicone edges (Comparative Example 4) while the image for conditioners with only nanoemulsion Si has more evenly distributed silicone clouds without distinct droplet boundaries (Comparative Example 5). Once the nanoemulsions and neat silicone are blended, the edges to the silicone droplets become blurry and the silicone signal becomes “diluted” (Inventive Example 1). It is believed this “clouding” effect on the neat silicone oil droplet is reducing excess deposition and improving hair free-flowing. This is showing there is some synergistic interaction between nanoemulsion and silicone oil, which gives rise to the light weight yet conditioning benefit.

### IFM and Pendulum Free Flow Results

All performance data are included in Table 2. IFM results indicate the smoothness of the hair surface, while Pendulum results demonstrate the free-flow property of hair. The present inventors have found that a product that provide both a low IFM result and a high pendulum result performs well. A conditioner made with just the Waro Y20875 nanoemulsion (Comparative Example 5) has a high pendulum result with very good free-flowing property, but its high IFM result shows the hair surface is not smooth enough. On the contrary, a conditioner made with neat silicone oil alone (Comparative Example 4) has a low IFM result, but its pendulum result is also low, showing hair is smooth but not free flowing. Surprisingly, conditioners made with a blend of Waro Y20875 and silicone oil comply with both favorable boundary conditions (Inventive Example 1 & 2). Not only is the free-flow benefit greatly elevated, but also the hair surface smoothness is largely retained. This indicates that the blends of silicone nanoemulsion and neat silicone oil modify the hair surface without much deposition onto the hair.

TABLE 2
Performance Results Data Table

| | 5 Cycle | 5 Cycle |
|---|---|---|
| Sample Name | IFM | Pendulum |
| Preferred Performance Boundary | <70 (smooth) | >1 (free- |
| Limit | | flowing) |
| Inventive Example 1 | 60.34 | 1.533 |
| Inventive Example 2 | 66.47 | 2.02 |
| Inventive Example 3 | 51.150 | 0.960 |
| Comparative Example 4 | 48.040 | 0.639 |
| Comparative Example 5 | 84.760 | 1.943 |
| Comparative Example 6 | 56.284 | 0.619 |

The inventors find products with IFM results below 70 relatively smooth, and pendulum results above 1 relatively free-flowing. With the preferred performance range and results from both inventive and comparative examples, the inventor calculated preferred blend ratio based on interpolation, as shown in Table 3.

TABLE 3
Preferred Silicone Ratio Range

| | Nano Si:Neat | 5 Cycle | 5 Cycle |
|---|---|---|---|
| Sample Name | Si Ratio | IFM | Pendulum |
| Inventive Example 2 | 75:25 | 66.47 | |
| Comparative Example 5 | 100:0 | 84.760 | |
| Interpolation for IFM Limit | 73:27 | 70 | |
| Inventive Example 3 | 25:75 | | 0.960 |
| Comparative Example 4 | 0:100 | | 0.639 |
| Interpolation for Pendulum Limit | 37:63 | | 1 |

Hence, once the nano-sized silicone to neat silicone ratio is decreased to below 37:63, the 5-cycle pendulum value reduces to below 1. This indicates there is a limit to the blending ratio for the free-flowing benefit. Similarly, when the nano-sized silicone to neat silicone ratio is increased to above 73:27, the 5-cycle IFM result increases to above 70, beyond which the hair surface is considered less smooth.

In addition, conditioners made with the blend with ADM8100E nanoemulsion (Comparative Example 6) is smooth but not free flowing, indicating not all nanoemulsions blending with neat silicone oil work to gain both benefits. The silicone contained inside ADM8100E is not a polyquaternium silicone.

A deposition study was done on silicones, fatty alcohols and cationic surfactants to understand the performance benefit. The silicone deposition data in Table 4 for Inventive Example 1 and Comparative Examples 4, 5, and 6 shows why the blend of Waro Y20875 nanoemulsion with silicone oil still provides a free-flowing benefit. The amount of deposition is greatly reduced from conditioners with the neat silicone oil, and it didn't accumulate with multi-cycle washes like the neat silicone oil. It also explained why conditioner with ADM8100E didn't improve free-flow property. The amount of silicone deposition is huge, signifying nanosized silicone property matters.

Interestingly, the same effect is also observed for fatty alcohol and cationic surfactant deposition. The amount of FaOH and Quat-surfactant deposition for the inventive example 1 (blend of Waro Y20875 with neat TAS) is greatly reduced from comparative example 4 (neat TAS alone), comparable to using just nanoemulsion silicone (comparative example 5). On the other hand, blend of ADM8100E and neat TAS (comparative example 6) showed huge deposition and accumulation over multicycle for both fatty alcohol and quat-surfactant.

TABLE 4
Deposition Results for Examples

| | Inventive Example 1 | | Comparative Example 4 | | Comparative Example 5 | | Comparative Example 6 | |
|---|---|---|---|---|---|---|---|---|
| Cycle # | 1 | 5 | 1 | 5 | 1 | 5 | 1 | 5 |
| Silicone | 270 | 555 | 917 | 2571 | 292 | 299 | 1126 | 5888 |
| (ug/g hair) | | | | | | | | |
| FaOH | 313 | 413 | 536 | 827 | 253 | 434 | 390 | 986 |
| (ug/g hair) | | | | | | | | |
| Quat | 214 | 195 | 332 | 437 | 178 | 192 | 195 | 452 |
| (ug/g hair) | | | | | | | | |

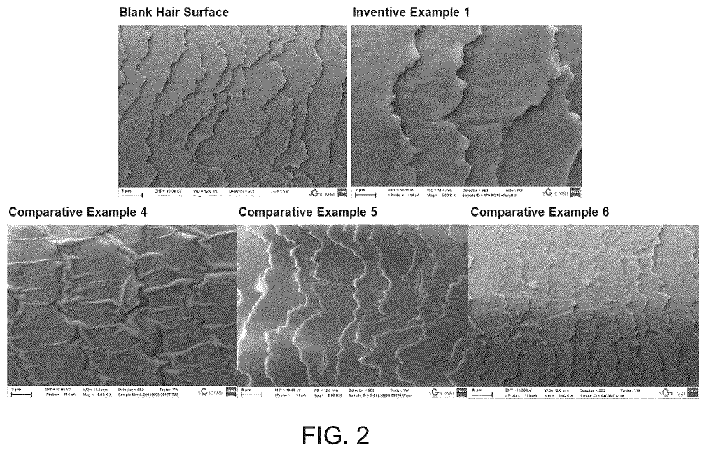

SEM images of hair treated with the example formulas are shown in FIG. 2. Except for the blank hair image, all hair surfaces are treated with the example formulas for 5 cycles. For Waro Y20875 (Comparative Example 5) and its blend with neat silicone oil (Inventive Example 1), hair cuticles are distinct and no obvious material deposition can be observed, while for neat silicone oil (Comparative Example 4) and ADM8100E (Comparative Example 6) treated hair surfaces, thick layers of deposition can be observed on the hair strands.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioner composition comprising
(a) a blend of a polyquaternium silicone nanoemulsion and a silicone oil, wherein
(i) a polyquaternium silicone contained in the nanoemulsion has a particle size of about 1 nm to about 100 nm and comprises silicone blocks of about 80 to about 250 siloxane units,
(ii) the silicone oil has a particle size of about 1 micron to about 100 microns, and
(iii) a weight ratio of the polyquaternium silicone to the silicone oil is about 1:2 to about 3:1
(b) a cationic surfactant;
(c) a fatty alcohol; and
(d) an aqueous carrier, wherein the fatty alcohol, cationic surfactant, and aqueous carrier form a gel network.

2. The composition of claim 1, wherein the polyquaternium silicone is a polyorganosiloxane compound comprising one or more quaternary ammonium groups.

3. The composition of claim 1, wherein the polyquaternium silicone comprises silicone blocks of about 100 to about 225 siloxane units.

4. The composition of claim 3, wherein the polyquaternium silicone comprises silicone blocks of about 125 to about 200 siloxane units.

5. The composition of claim 1, wherein the silicone oil comprises nitrogen.

6. The composition of claim 1, wherein the silicone oil comprises a dimethicone, dimethiconol, quaternary ammonium silicone, quaternary ammonium polyether silicone, or a combination thereof.

7. The composition of claim 1, wherein the weight ratio of polyquaternium silicone to silicone oil is between 37:63 and 73:27.

8. The composition of claim 1, wherein the polyquaternium silicone has a particle size of about 1 nanometer to about 60 nanometers.

9. The composition of claim 1, wherein the polyquaternium silicone nanoemulsion has a particle size of about 20 nanometers to about 40 nanometers.

10. The composition of claim 1, wherein the composition comprises about 1% to about 30%, by weight of the composition, of the blend of polyquaternium silicone nanoemulsion and silicone oil.

11. The composition of claim 1, wherein the composition comprises about 0.5% to about 20%, by weight of the composition, of the polyquaternium silicone nanoemulsion.

12. The composition of claim 1, wherein the composition comprises about 0.1% to about 10%, by weight of the composition, of the silicone oil.

13. The composition of claim 1, wherein the composition comprises about 0.5% to about 10%, by weight of the composition, of the cationic surfactant.

14. The composition of claim 1, wherein the composition comprises about 0.5% to about 15%, by weight of the composition, of the fatty alcohol.

15. The composition of claim 1, wherein the composition comprises about 45% to about 98%, by weight of the composition, of the aqueous carrier.

16. The composition of claim 1, wherein the polyquaternium silicone nanoemulsion further comprises at least one emulsifier and at least one solvent.
